(19) United States
(12) Patent Application Publication
(10) Pub. No.: US 2014/0188043 A1
(43) Pub. Date: Jul. 3, 2014

(54) Balloon Catheter Having Purge Hole

(71) Applicant: Fuji Systems Corporation, Tokyo (JP)

(72) Inventor: Toshio Shibahara, Fukushima (JP)

(73) Assignee: Fuji Systems Corporation, Tokyo (JP)

(21) Appl. No.: 14/091,687

(22) Filed: Nov. 27, 2013

(30) Foreign Application Priority Data
Dec. 27, 2012 (JP) 2012-283974

(51) Int. Cl. A61M 25/10

(52) U.S. Cl. CPC A61M 25/10
USPC 604/96.01

(57) ABSTRACT

A balloon catheter having a purge hole according to the present invention is configured with a duplex tube structure of an inner tube and an outer tube both of which having an opening at the distal end thereof. A balloon is provided to stretch over the section between the distal ends of the inner tube and the outer tube. A purge hole is provided on the inner tube covered by the balloon, through which the remaining air inside the balloon lumen and the balloon is discharged to a main lumen formed in the inner tube when the balloon is expanded, and which is closed by the balloon when the balloon is contracted.

### BACKGROUND OF THE INVENTION

The present invention relates to a balloon catheter having a purge hole, more particularly, to an improvement of a balloon catheter used in a blood vessel in which the remaining air in a balloon lumen and a balloon is discharged to the outside of the catheter through a purge hole when the balloon, used for cutting off blood flow, expanding a blood vessel, or the like at a given portion, expands.

An example of such balloon catheter being used includes: (1) a guiding catheter; (2) a stenosed region expansion catheter; and (3) a liquid medicine injection catheter.

(1) The guiding catheter is a catheter indwelled in a blood vessel from a puncture hole from which a catheter is inserted to a vicinity of a lesion. Generally, in an endovascular repair, a surgical instrument such as a stent advances and retreats many times in a region from a puncture hole to a lesion. When the surgical instrument advances and retreats directly inside the blood vessel, the surgical instrument contacts the blood vessel wall or the like and may damage the blood vessel. When a surgical instrument such as a stent advances and retreats in the region from the puncture hole to the lesion via the guiding catheter, the operation can be carried out without a surgical instrument making a direct contact with a blood vessel wall or the like.

(2) The stenosed region expansion catheter is a catheter used for curing angiostenosis (a disease in which a cholesterol, a fat, or a thrombus accumulates and narrows a vascular lumen). A blood lumen is extended by expanding a balloon in the stenosed region of the vascular lumen.

(3) The liquid medicine injection catheter is a catheter used for injecting a liquid medicine in a blood vessel.

A typically known conventional balloon catheter 01 is illustrated in FIGS. 8 and 9. The balloon catheter 01 is configured with a duplex tube structure of an inner tube 02 which is an inner tubular member and an outer tube 03 which is an outer tubular member. The distal end of the outer tube 03 is arranged in a position offset from a distal end of the inner tube 02 by a given distance toward the rear end. A balloon 05 is provided to stretch over the section between the distal ends of the inner tube 02 and the outer tube 03. A balloon lumen 06 used for expanding and contracting the balloon 05 with sterilized water is provided between the inner tube 02 and the outer tube 03 with the distal end of the balloon lumen 06 opened to the inner tube 02.

Typically, as a preparation for using the balloon catheter 01 safely in a blood vessel, the air inside the balloon lumen 06 and the balloon 05 is discharged outside the catheter before using the catheter. By this preparation, the air coming into the blood vessel to cause an air embolism is prevented, in case when the balloon 05 burst. In order to easily remove the air, a small hole for removing air, that is, a purge hole 08, is provided on the outer side of the balloon 05, that is, the outer tube 03 provided in the rear end side relative to the balloon 05, so as that the air is discharged through the purge hole 08. As for the balloon catheter 01, there may be a concern that the injected sterilized water might leak from the purge hole 08 when the balloon 05 is expanded. However, since the hole is extremely small, there is no possibility of the leaking of the sterilized water.

However, as illustrated in the drawing, the purge hole 08 for removing air is permanently open. Therefore, there exists a problem that when a negative pressure is applied to contract the balloon 05 with the catheter indwelled in the blood vessel, the blood is suctioned through the purge hole 08 and the blood may clog and cause choking in the balloon lumen 06.

The related patent document includes Patent Document 1 (Japanese Patent Laid-Open Publication No. 2005-103120) and Patent Document 2 (Japanese Patent Laid-Open Publication No. 2011-072438). A balloon catheter 1 disclosed in Patent Document 1 has a duplex tube structure configured with an inner tube 9 and an outer tube 21 as illustrated in FIG. 3 in detail, which is similar to the structure of the conventional balloon catheter 01. A balloon 7 is provided so as to stretch over the section between the distal ends, and a purge hole 24 is provided at the distal end of the outer tube adjoining the balloon. A purge hole cover 33 which covers the purge hole 24 and can be opened and shut is provided to constitute a one-way valve together with the purge hole 24. As mentioned above, an additional purge hole cover is required. Further, as for Patent Document 2, an expandable resin 4 is arranged in the distal end side of an inflation lumen 3 as illustrated in FIG. 1 in detail. A hole 5 which links the inflation lumen 3 with the outside of the catheter is provided to the expandable resin 4, and a bubble remaining inside the balloon can be removed through the hole 5. However, an additional expandable resin is required.

### SUMMARY OF THE INVENTION

With regard to such existing problem as described above, the object of the present invention is to provide a balloon catheter in which a blood is not sucked through the purge hole and a choking caused by clogging of blood in a balloon lumen can be prevented even when a negative pressure is applied to contract the balloon in a blood vessel, without using any conventional member such as a purge hole cover and an expandable resin as described in Patent Documents 1 and 2.

To achieve the object, the balloon catheter according to the present invention is configured with a duplex tube of an inner tube and an outer tube both of which having an opening at a distal end thereof, in which: the distal end of the outer tube is arranged in a position offset from the distal end of the inner tube by a given distance toward the rear end; a balloon is provided so as to stretch over the section between the distal ends; and a balloon lumen for expanding and contracting the balloon with sterilized water is provided between the tubes, with the distal end of the balloon lumen opened to the inner tube. The feature of the balloon catheter according to the present invention is that a purge hole, through which the remaining air inside the balloon lumen and the balloon is discharged to the main lumen formed in the inner tube when the balloon is expanded, and which is closed by the balloon when the balloon is contracted, is provided on the inner tube covered by the balloon. Further, another balloon catheter according to the present invention includes a tubular catheter main body having an opening at a distal end thereof with a main lumen formed in the axial direction, in which: a balloon is provided at the distal end of the catheter main body; and a balloon lumen for expanding and contracting the balloon by injection of sterilized water is formed in the catheter main body in the axial direction. The feature of the balloon catheter according to the present invention is that a purge hole is provided on the catheter main body covered by the balloon, through which the remaining air inside the balloon lumen and the balloon is discharged to the main lumen of the catheter main body when the balloon is expanded, and which is closed by the balloon when the balloon is contracted.

According to the present invention having such configuration, the purge hole is closed by the contracting balloon when a negative pressure is applied to contract the balloon in the blood vessel, whereby the blood suction through the purge hole can be prevented. Consequently, in addition to the prevention of choking caused by clogging of blood in the balloon lumen, no such member like a purge hole cover or an expandable resin is necessary, thereby providing an excellent effect to simplify the configuration.

### BRIEF DESCRIPTION OF THE DRAWINGS

FIG. 1 is an essential front view illustrating a balloon catheter having a purge hole in a state in which a balloon is expanded according to an embodiment of the present invention;

FIG. 2 is an enlarged view of the portion A in FIG. 1;

FIG. 3 is an essential front view illustrating a state in which a balloon is contracted;

FIG. 4 is an essential front view illustrating a balloon catheter having a purge hole in a state in which a balloon is expanded according to another embodiment of the present invention;

FIG. 5 is an essential front view illustrating a state in which a balloon is being contracted;

FIG. 6 is an essential front view illustrating a state in which a balloon is contracted;

FIG. 7 is an essential front view illustrating an exemplary variation in a state in which a balloon is contracted;

FIG. 8 is an essential front view illustrating a typically known conventional balloon catheter having a purge hole in a state in which a balloon is expanded; and

FIG. 9 is an essential front view illustrating a state in which a balloon is contracted.

### DETAILED DESCRIPTION OF THE INVENTION

A balloon catheter having a purge hole according to an embodiment of the present invention will be described below referring to the drawings.

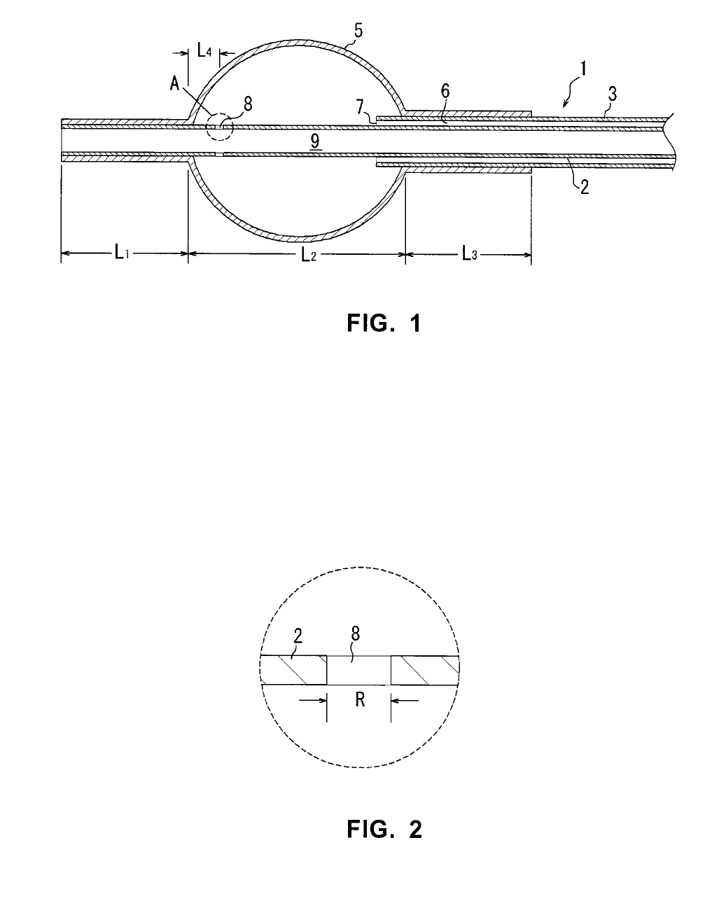

Referring to FIG. 1 to FIG. 3, a balloon catheter 1 used in a blood vessel is formed of a biocompatible material such as a thermoplastic resin which may be PP, PE, and PU, and a silicone gum, and configured with a duplex tube of an inner tube 2 and an outer tube 3, both of which have an opening at the distal end thereof. The inner tube 2 and the outer tube 3 are arranged so as the distal end of the outer tube 3 to be in a position offset from the distal end of the inner tube 2 by a given distance toward the rear end. A balloon 5 is provided so as to stretch over the periphery of the section between the distal ends of the inner tube 2 and the outer tube 3. Further, an annular balloon lumen 6 is provided between the inner tube 2 and the outer tube 3 with the distal end of the balloon lumen 6 opened to the inner tube 2 to form a water inlet 7. The balloon 5 is expanded and contracted by sterilized water injected from the water inlet 7 through the balloon lumen 6.

Two purge holes 8 are provided in the distal end side of the inner tube 2 covered by the balloon 5 as described above, particularly at a location in the distal end side along the axial direction from the middle of the balloon 5, and at a location close to the end of the inner tube 2, so as to be apart from each other in the circumferential direction by 180 degrees. The number of the purge holes 8 is not limited to two. The number may be one, three, or more if necessary. When the balloon 5 expands, the remaining air inside the balloon lumen 6 and the balloon 5 is discharged through the purge hole 8 into a main lumen 9 formed in the inner tube 2. When the balloon 5 contracts, the purge hole 8 is closed by the balloon 5. The purge hole 8 is provided in the distal end side of, and at the location close to the end of, the inner tube 2 so that the purge hole 8 can easily be closed by the balloon 5 when the balloon 5 is contracted.

As illustrated in FIG. 1, the lengths of overlapping portions L1 and L3 (L1=L3) provided in the front side and the rear side of the balloon 5 in the axial direction, respectively, are determined to be smaller than a balloon effective length L2 but as large as, or larger than, a half of the balloon effective length L2, so as to stably fix the balloon 5. Further, the purge hole 8 is provided so as the center thereof to be arranged in the location which is L4 from the boundary of the overlapping portion L1 of the balloon 5. Specifically, the location is a one-eighth of the L2 from the boundary. The purge hole 8 need not be arranged in the location illustrated in the drawing as long as the purge hole 8 can be closed by the contracted balloon 5. For example, the purge hole 8 may be arranged in other locations such as a location a little bit closer to the middle of the balloon 5. Further, a hole diameter R of the purge hole 8 may preferably be about 20 μm (see FIG. 2).

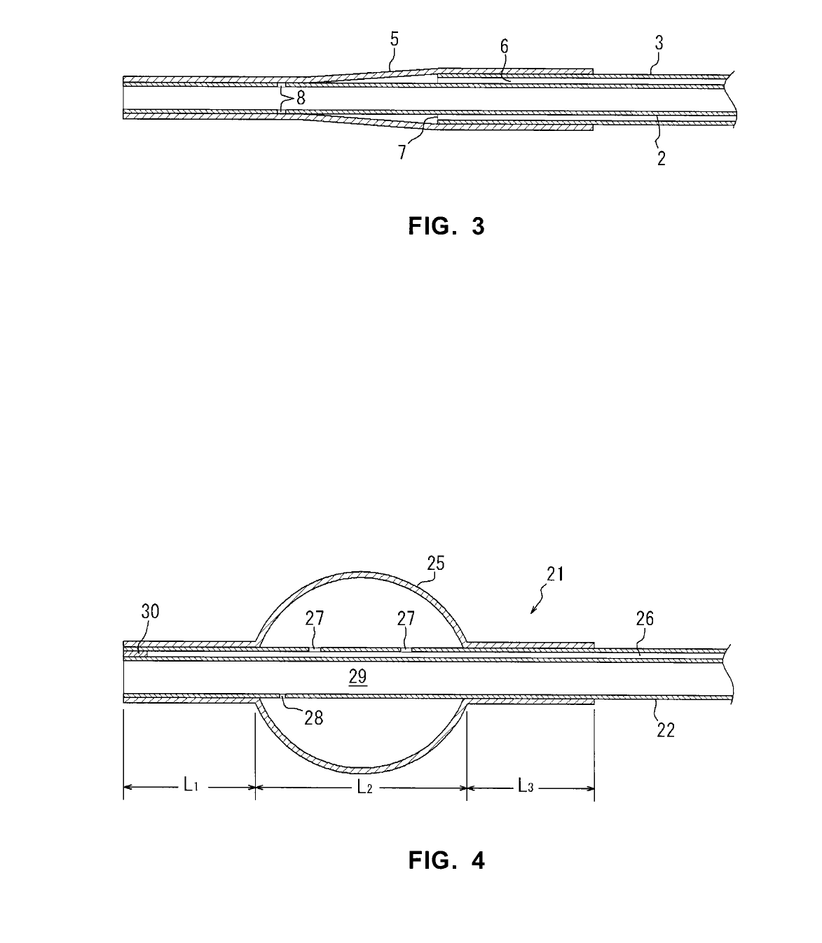

The effect of the balloon catheter 1 will be described. When, as a preparation before using, the sterilized water is injected through the balloon lumen 6 and from the opening at the distal end thereof, that is, the water inlet 7 so as to discharge the air inside the balloon lumen 6 and the balloon 5, the balloon 5 gradually expands by the injection of the sterilized water to be in an expanded state as illustrated in FIG. 1. During the period until the balloon 5 is expanded to the expanded state, the remaining air inside the balloon lumen 6 and the balloon 5 is discharged through the purge hole 8 into the main lumen 9, and further discharged outside the catheter through the opening at a distal end of the main lumen 9. On the other hand, when a negative pressure is applied to the catheter to contract the balloon 5, the balloon 5 gradually contracts to be in a contracted state as illustrated in FIG. 3. During the period until the balloon 5 is contracted to the contracted state, the contracting balloon 5 covers and sticks on the purge hole portion. Consequently, the purge hole 8 is closed as illustrated in the drawing so that the balloon 5 acts as a one-way valve, which can prevent the suction of body fluid, blood, or the like. In the contracted state of the balloon 5 as described above, a gap, having a height of the step formed by the inner tube 2 and the outer tube 3, is created in the rear end side of the balloon 5, that is, in the region from the middle portion of the balloon 5 to the rear overlapping portion. Although, this does not cause any problem to the closing of the purge hole 8 since the purge hole 8 is not located within the range of the gap.

By providing the purge hole 8 in the distal end side of the inner tube 2 which is covered by the balloon 5 as described above, the existing problem of blood suctioning through the purge hole 8 under a negative pressure can be solved.

In other words, while the blood is suctioned through the purge hole 08 and clogs to cause choking in the balloon lumen 06 in a conventional technique, the purge hole 8 is closed by the contracting balloon 5 so that the blood suctioning is prevented, when the balloon catheter 1 is used. Therefore, the clogging of the blood and choking in the balloon lumen 6 can be prevented. Further, as for the configuration, it is simply required that the purge hole 8 is formed in the distal end side of the inner tube 2 covered by the balloon 5. Therefore, the configuration is simple since no additional member is required to be provided.

FIGS. 4 to 6 illustrate another embodiment. A balloon catheter 21 of the embodiment is formed of a material similar to that of the balloon catheter 1, and includes a tubular catheter main body 22 having an opening at the distal end thereof. A balloon 25 is provided on the periphery of the distal end of the catheter main body 22. Further, a main lumen 29 is formed inside the catheter main body 22 of the catheter main body 22 in the axial direction, and a balloon lumen 26 for injecting water to expand and contract the balloon 25 is formed on the wall of the catheter main body 22 in the axial direction. Two water inlets 27 are provided on the catheter main body 22 covered by the balloon 25 in the axial direction, so as to link the balloon lumen 26 and the inside of the balloon 25.

Further, a purge hole 28 is provided in the distal end side of, and at the location close to the end of, the catheter main body 22 covered by the balloon 25. When the balloon 25 expands, the remaining air inside the balloon lumen 26 and the balloon 25 is discharged through the purge hole 28 into a main lumen 29 of the catheter main body 22. When the balloon 25 contracts, the purge hole 28 is closed by the balloon 25. The arranged location and the number of the purge hole 28 are merely described as a preferable example. Any location and number can be selected for carrying out the present invention. A distal end plug 30 of the balloon lumen 26 is assembled to the distal end of the balloon lumen 26, and prevents the sterilized water flowing in the balloon lumen 26 from flowing further than the distal end plug 30 toward the distal end side. Other configurations such as overlapping portions in the front and rear sides of the balloon 25 are similar to those of the embodiment described above. The same numeral is used for the portion similar to that of the embodiment described above and the description of the portion is omitted.

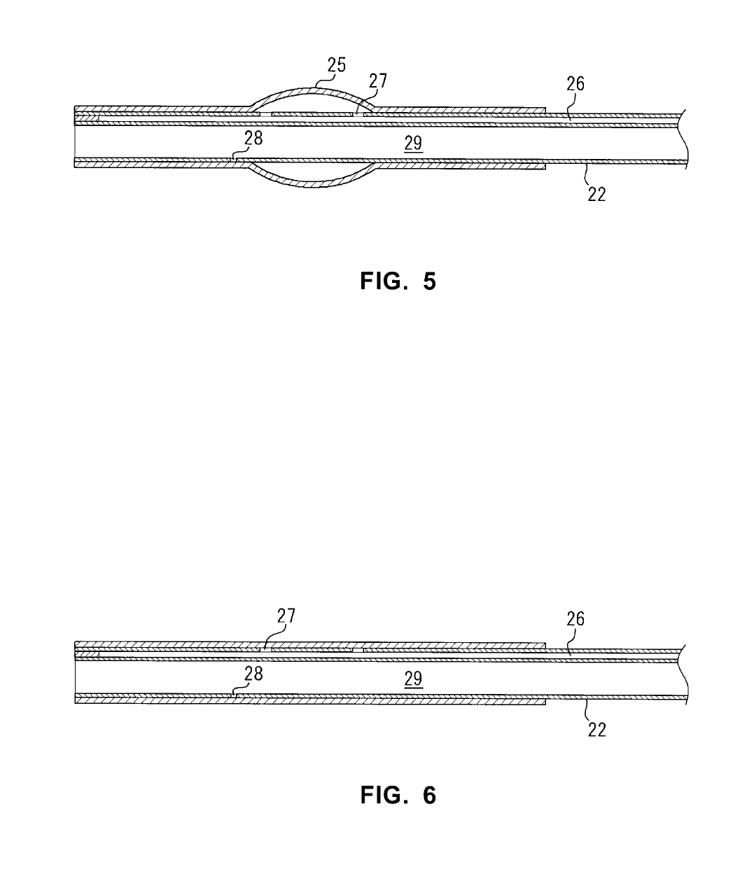

Now, the effect of the balloon catheter 21 will be described. When, as a preparation before using, the sterilized water is injected through the balloon lumen 26 and from the opening at the distal end thereof, that is, the water inlet 27 so as to discharge the air inside the balloon lumen 26 and the balloon 25, the balloon 25 gradually expands by the injection of the sterilized water to be in an expanded state as illustrated in FIG. 4. During the period until the balloon 25 is expanded to the expanded state, the remaining air inside the balloon lumen 26 and the balloon 25 is discharged through the purge hole 28 into the main lumen 29, and further discharged outside the catheter through the opening at the distal end of the main lumen 29. On the other hand, when a negative pressure is applied to the catheter 21 to contract the balloon 25, the balloon 25 gradually contracts to be in an intermediate state as illustrated in FIG. 5 and finally be in a contracted state as illustrated in FIG. 6. During the period until the balloon 25 is contracted to the contracted state, the contracting balloon 25 covers and sticks on the purge hole portion. Consequently, the purge hole 28 is closed so that the balloon 25 acts as a one-way valve, which can prevent the suction of body fluid, blood, or the like. In the contracted state of the balloon 25 as described above, the entire portion of the balloon 25 sticks on the periphery of the distal end of the catheter main body 22 without a gap so that the purge hole 28 is closed without a problem.

As apparent from FIG. 5 which illustrates the balloon 25 in an intermediate state of contraction, the purge hole 28 is already closed by the contracting balloon 25 before the balloon 25 reaches a completely contacted state as in FIG. 6, that is, just after a moment the balloon 25 started to contract. Therefore, it can be understood that the purge hole 28 is closed, so as to prevent blood suction or the like, at the intermediate state of contraction before the balloon 25 is completely contracted.

By providing the purge hole 28 in the distal end side of the catheter main body 22 which is covered by the balloon 25 as described above, the existing problem of blood suctioning through the purge hole 28 under a negative pressure can be solved.

In other words, while the blood is suctioned through the purge hole 08 and clogs to cause choking in the balloon lumen in a conventional technique, when the balloon catheter 21 is used, on the other hand, the purge hole 28 is closed by the contracting balloon 25 so that the blood suction is prevented. Therefore, the clogging of the blood and choking in the balloon lumen 26 can be prevented. Further, as for the configuration, it is simply required that the purge hole 28 is formed in the distal end side of the catheter main body 22 covered by the balloon 25. Therefore, the configuration is simple since no additional member is required to be provided.

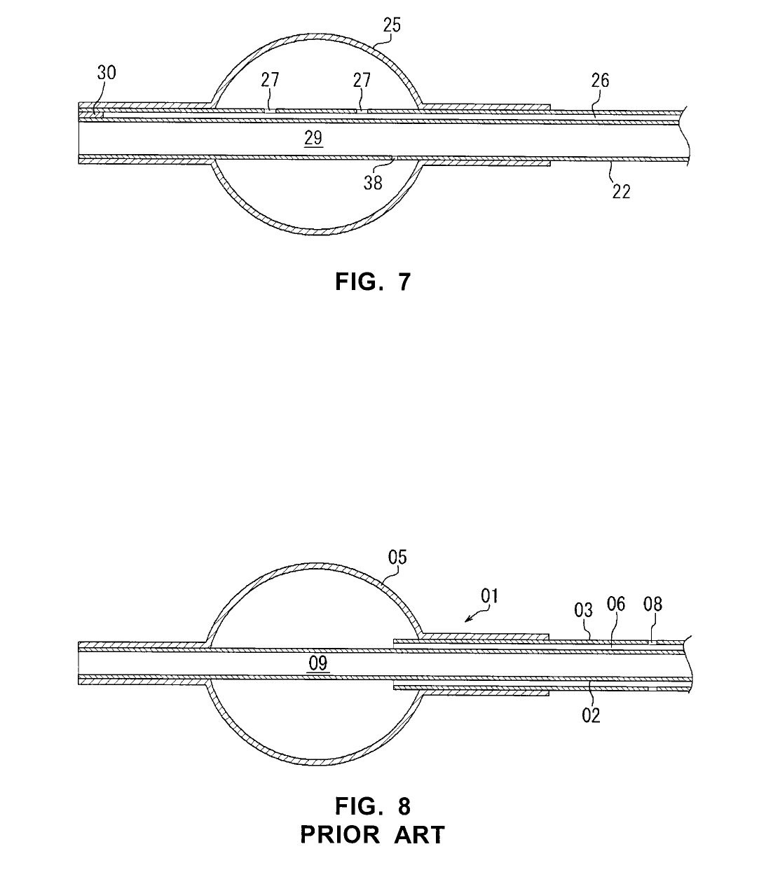

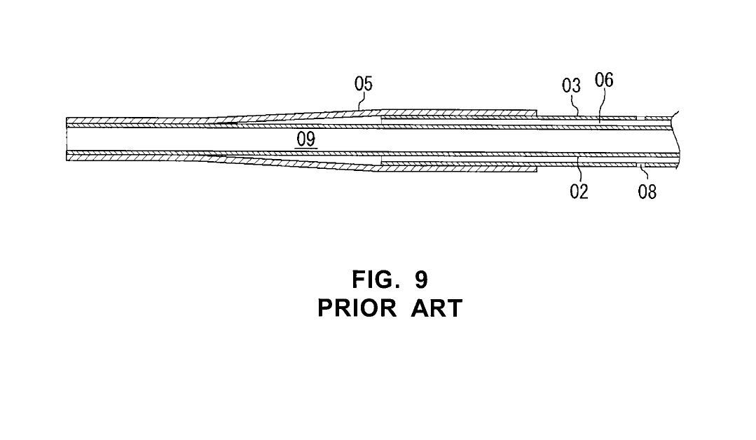

FIG. 7 illustrates an exemplary variation in which a purge hole 38 is provided in the rear end side of, and at the location close to the end of, the catheter main body 22 covered by the balloon 25. For such arranged location of the purge hole, the effect similar to the effect described above can be obtained. The contracting balloon 25 covers and sticks on the purge hole portion so as to close the purge hole 38, whereby the balloon 25 acts as a one-way valve, which can prevent the suction of body fluid, blood, or the like.

Each of the balloon catheters 1 and 21 described in the embodiment above is merely a preferable example. It goes without saying that any alteration and modification can be made for detailed designs without departing from the scope described in claims of the present invention.

1. A balloon catheter having a purge hole configured with a duplex tube structure of an inner tube and an outer tube both of which having an opening at a distal end thereof, in which the distal end of the outer tube is arranged in a position offset from the distal end of the inner tube by a given distance toward a rear end, a balloon is provided to stretch over a section between the distal ends of the inner tube and the outer tube, and a balloon lumen for expanding and contracting the balloon with sterilized water is provided between the inner tube and the outer tube with a distal end of the balloon lumen opened to the inner tube, wherein
a purge hole, through which the remaining air inside the balloon lumen and the balloon is discharged to a main lumen formed in the inner tube when the balloon is expanded, and which is closed by the balloon when the balloon is contracted, is provided on the inner tube covered by the balloon.

2. The balloon catheter having a purge hole according to claim 1, wherein the purge hole is provided in a distal end side of the inner tube covered by the balloon.

3. A balloon catheter having a purge hole including a tubular catheter main body having an opening at a distal end thereof with a main lumen formed in an axial direction, in which a balloon is provided at a distal end of the catheter main body, and a balloon lumen for expanding and contracting the balloon by injection of sterilized water is formed in an axial direction of the catheter main body, wherein
a purge hole is provided on the catheter main body covered by the balloon, through which a remaining air inside the balloon lumen and the balloon is discharged to the main lumen of the catheter main body when the balloon is expanded, and which is closed by the balloon when the balloon is contracted.

4. The balloon catheter having a purge hole according to claim 3, wherein the purge hole is provided in a distal end side or a rear end side of the catheter main body covered by the balloon.
